(19) United States
(12) Patent Application Publication
(10) Pub. No.: US 2009/0110632 A1
(43) Pub. Date: Apr. 30, 2009

(54) BIOLOGICAL MATERIALS AND USES THEREOF

(76) Inventors: Robert Young, London (GB); David Jones, London (GB); Nigel Courtenay-Luck, London (GB); Dylan Glub, Christchurch (NZ); Benjamin Doran, London (GB); Paul Michael Rogers, London (GB); Babu Dhokia, London (GB); Christy Ritchie, London (GB); Rakesh Verma, London (GB)

Correspondence Address: JAECKLE FLEISCHMANN & MUGEL, LLP, 190 Linden Oaks, ROCHESTER, NY, 14625-2812, US

(21) Appl. No.: 12/067,905

(22) PCT Filed: Sep. 22, 2006
(86) PCT No.: PCT/GB06/03541

§ 371 Date: Nov. 14, 2008

(30) Foreign Application Priority Data
Sep. 23, 2005 (GB) 0519398.2

(51) Int. Cl. A61K 51/00; C07K 16/18; C12N 15/11; C12N 15/00; A61K 38/43; C40B 30/04; C12N 5/06; A61K 39/395; A61K 31/7088

(52) U.S. Cl. 424/1.11; 530/387.3; 536/23.53; 435/320.1; 435/328; 424/133.1; 424/135.1; 514/44; 424/94.1; 506/9

(57) ABSTRACT

There is provided by the invention a modified antibody molecule which selectively binds to a specific target, the antibody molecule being modified at, at least one amino acid residue that forms part of a glycosylation site in the variable region of an unmodified parent antibody molecule, characterised in that the modified antibody is not glycosylated at the previous glycosylation site of which the amino acid modification forms part and the modified antibody exhibits a greater binding affinity for the specific target than the unmodified parent antibody molecule. There is also provided nucleotide sequences, amino acid sequences and expression vectors encoding the modified antibodies, and uses thereof.

The present invention relates to antibodies, antibody fragments and antibody derivatives possessing improved binding properties.

Antibodies are currently used in many clinical applications, including for cancer therapy. These include unconjugated antibodies that exert their effect through a variety of mechanisms including recruitment of host immune functions or blocking receptor-ligand interactions, as well as antibodies coupled to cytotoxic agents or radionuclides.

The first antibodies used clinically were murine antibodies, which had the potential to elicit an immune response in the patient, and were less efficient than human antibodies in the recruitment of human immune effector cells. To resolve this, murine antibody constant regions were first replaced with human constant regions, so-called chimeric antibodies. For the next generation of engineered, antibodies, the majority of murine amino acids were exchanged for the equivalent human sequence, leaving only a few murine sequences, largely in the antigen binding regions of the antibody.

Antibodies are glycoproteins possessing an oligosaccharide attached to each heavy chain constant region. These glycosylating play a role in binding complement, binding IgG receptors on effector cells and stabilising the antibody.

Many naturally occurring antibodies also contain additional oligosaccharide molecules in the variable region of the antibody.

Many clinical applications, such as radioimmunoimaging, radioimmunotherapy, or administration of recombinant cytotoxic fusion proteins, favourably employ antibody fragments or small antigen binding molecules such as Fab molecules or multivalent derivatives. In some cases, these smaller antibody fragments or antigen binding molecules possess advantages over the use of whole antibodies (wild type or humanised) in the IgG format. For example, in contrast to whole immunoglobulins, scFv fragments are capable of penetrating solid tumour tissue efficiently (Yakota, T. et al. (1992) Cancer Res 52:3402) and are rapidly cleared from the circulation (Milenic, D. E. et al. (1991) Cancer Res 51:6363). An alternative way to improve the properties of whole antibodies is to optimise certain properties such as affinity.

It is of paramount importance in clinical applications that an antibody or fragment exhibits sufficient affinity to the target antigen while possessing a high degree of stability and a sufficiently long half-life to allow the antibody to reach, its target and remain active for a clinically acceptable period. Failure to meet these major requirements can result in insufficient enrichment of antibodies or fragments thereof in xenografted solid tumours in immunodeficient mice, as shown in Adams, G. P, et al. (1998) Cancer Res 58:485 and Willuda, J. et al. (1999) Cancer Res 59:5758, thus hampering future clinical applications.

Previously known antibodies, for example HMFG1, possess one or more binding properties e.g. binding affinity, that are not optimised. Therefore, the present invention seeks to solve this problem by providing an antibody molecule exhibiting enhanced binding properties.

The MUC-1 gene product, the membrane mucin glycoprotein (polymorphic epithelial mucin or PEM) has been shown to be over-expressed in most adenocarcinomas (Taylor-Papadimitriou et al. (1999) Biochim Biophys Acta 1455:301.). MUC-1 over-expression has been widely associated with poor prognosis in patients with colorectal and gastric carcinoma (Baldus, S. E., et al. (2002) Histopathology 40:440; Utsunomiya, T. et al. (1998) Clin Cancer Res 4:2605). MUC-1 has been found more recently to be over-expressed in a variety of haematological malignancies including acute myelogous leukaemia, chronic lymphocytic leukaemia, and multiple myeloma (Brossart, P. et al. (2001) Cancer Res 61:6846).

The glycosylation of MUC-1 glycoprotein in cancer cells is distinct from that expressed in healthy tissue (Hanisch, F. G., and Mullet, S. (2000) Glycobiology 10:439). As such, tumour-associated mucin glycoproteins have been identified as representing a valuable target for diagnostic and therapeutic approaches using monoclonal antibodies (mAbs).

Several mAbs have been raised against the highly conserved immunogenic MUC-1 core region possessing tandem repeats of 20 amino acids in the extracellular portion of the MUC-1 glycoprotein (Gendler, S. et al. (1998) J Biol Chem 263:12820). These mAbs include HMFG1 which recognises a MUC-1 epitope with high selectivity (Taylor-Papdimitriou, J. et al. (1981) Int J Cancer 28:17).

HMFG1 is internalised by the cell after it has bound its target antigen (Aboud-Pirak, E. et al. (1988) Cancer Res 48:3188). Therefore, HMFG1 provides a valuable tool for the selective delivery of cytotoxic agents into tumour cells. Consequently, a ⁹⁰Y-murineHMFG1 radioimmunoconjugate was employed in a phase I-II clinical trial in patients with advanced ovarian cancer in an adjuvant setting. Intraperitoneal administration of a single dose of the reagent resulted in a >10 year long term survival of 78% of these patients (Epenetos, A. A. et al. (2000) Int J Gynecol Cancer 10:44).

A humanised version of HMFG1, designated huHMFG1, was generated by grafting the murine antigen-binding site onto human frameworks (Verhoeyen, M. E, et al. (1993) Immunology 78:364). huHMFG1, was shown to retain the antigen affinity and same selectivity as the rodent ancestor.

To exploit the potential advantages of antibody fragments, huHMFG1 has also been reformatted into an scFv fragment. The variable heavy (V_{H}) and variable light (V_{L}) domains of the antibody are involved in antigen recognition; a fact first recognised by early protease digestion experiments.

That antigenic selectivity is conferred by variable domains and is independent of the constant domains is known from experiments involving the bacterial expression, of antibody fragments, all containing one or more variable domains. These molecules include Fab-like molecules (Better et al. (1988) Science 240, 1041); Fv molecules (Skerra et al. (1988) Science 240, 1038); single-chain Fv (ScFv) molecules where the V_{H} and V_{L} partner domains are linked via a flexible oligopeptide (Bird et al. (1988) Science 242, 423; Huston et al. (1988) Proc. Natl. Acad. Sci USA 85, 5879) and single domain antibodies (dAbs) comprising isolated V domains (Ward et al. (1989) Nature 341, 544). A general review of the techniques involved in the synthesis of antibody fragments which retain their selective binding sites is to be found in Winter & Milstein (1991) Nature 349, 293-299.

One example of a variable region glycosylation site has been demonstrated in the HMFG1 antibody which has now been shown to possess an N-linked glycosylation site in the variable (antigen-binding) region at the asparagine-amino acid residue at position 56 (Asn56 or N56). Analysis has indicated that this site is at least partially glycosylated.

We show that modification of antibodies at a variable region glycosylation site so as to prevent glycosylation can surprisingly influence the binding properties of the antibody, and in particular binding affinity.

In a first aspect of the invention there is provided a modified antibody molecule which selectively binds to a specific target, the antibody molecule being modified at, at least one amino acid residue that forms part of a glycosylation site in the variable region of an unmodified parent antibody molecule, characterised in that the modified antibody is not glycosylated at the previous glycosylation site of which the amino acid modification forms part and the modified antibody exhibits a greater binding affinity for the specific target than the unmodified parent antibody molecule.

The affinity of the modified antibody molecule can be measured and compared using the methods described in example 3. The methods of example 3 measure the relative affinity of the modified antibody for the specific target in comparison to the unmodified parent antibody

The glycosylation of a particular amino acid residue can be predicted and identified using the methods of the examples, in particular, example 1.

Preferably the amino acid that has been modified in the unmodified parent antibody molecules is asparagine (Asn or N).

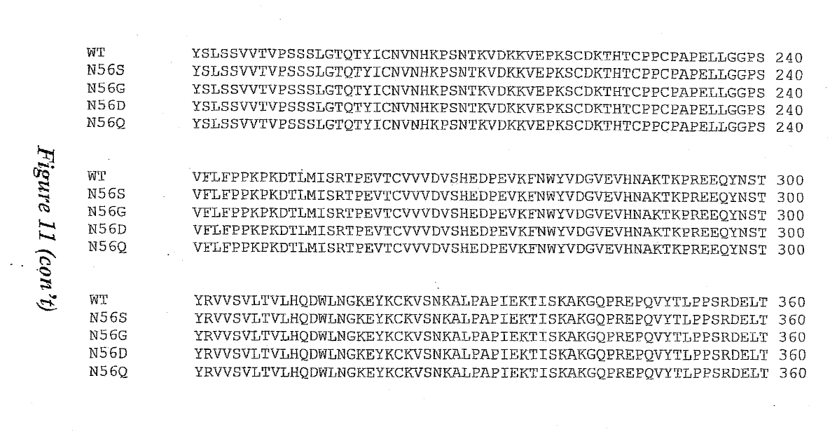

Conveniently, the site of the modification is the amino acid residue V_{H}56 of FIG. 11 or the corresponding residue in another antibody molecule.

The position of amino acid residues corresponding to the V_{H}56 amino acid residue of FIG. 11 is defined by its position in the secreted heavy chain (the fifty sixth residue of the mature heavy chain with signal peptide removed). The same residue can be identified in any given antibody or antibody fragment identified by the KABAT numbering system. The KABAT system can be accessed by submitting the Fv protein sequence online at http://www.bioinf.org.uk/abs/seqtest.html. The server for this site aligns the submitted sequence to all KABAT database entries and makes the accurate numbering of residues. Also, any “unusual” residues (i.e. occurrence at a given position <1%) are reported. Using this method the glycosylated asparagine residue of interest in HMFG1 is located at KABAT number 55 (due to an inserted residue in HMFG1). The sequences can also be aligned manually according to the method of Kabat et al. (1991) Sequences of Proteins of immunological Merest. NTH publication no. 91-3242.

Conveniently the specific target is the MUC-1 gene product.

Preferred modifications result in amino acids with small side chains.

Preferably the modification results in the amino acid, glycine. Alternative preferences are cysteine or alanine.

Conveniently the modified antibody molecule has a binding selectivity equivalent to anti-HMFG monoclonal antibody (HMFG1). Preferably the antibody that has been modified is HMFG1.

Equivalence of binding selectivity can be measured using methods described in example 3.

Alternatively the modified antibody molecule is a single chain antibody and may be a Fab, ScFv or a diabody.

The mutant scFv may also be further engineered into a bivalent diabody. The diabody molecule may exhibit improved pharmacokinetics and tumour retention properties (Adams et al., Br J Cancer 77, 1405-1412, 1998).

Conveniently the modified antibody molecule is humanised.

Humanised antibodies are suitable for administration to humans without invoking an immune response by the human against the administered immunoglobulin. Humanised forms of antibodies are intact immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanisation can be performed following the method of Winter and co-workers (Jones et al., Nature, 321:522-525 (1986); Riechmann et al., Nature, 332:323-327 (1988); Verhoeyen et al., Science, 239:1534-1536 (1988)). In some instances, Fv framework residues of the human immunoglobulin are replaced by corresponding non-human residues. Humanised antibodies can also comprise residues that are found neither in the recipient antibody nor in the imported CDR or framework sequences. In general, the humanised antibody will comprise substantially all of at least one, and typically two, variable regions, in which all or substantially all of the CDR regions correspond to those of a non-human immunoglobulin and all or substantially all of the framework regions are those of a human immunoglobulin consensus sequence. The humanised antibody optimally also will comprise at least a portion of an immunoglobulin constant region (Fc), typically that of a human immunoglobulin (Jones et al., 1986; Riechmann et al., 1988; and Presta, Curr. Op. Struct, Biol, 2:593-596 (1992)). Alternatively the antibody may be a chimeric antibody.

In an alternative embodiment of the first aspect of the invention the antibody may be considered to be the amino acid sequence of either of both the light and heavy chain polypeptides making up the three-dimensional antibody.

In a second aspect of the invention is provided a nucleic acid having a nucleotide sequence encoding the modified antibody molecule according to the first aspect of the invention.

The nucleotide sequence can be found using any common sequencing technique including the use of automatic sequence machines. For examples of sequencing methods, see Sambrook and Russell (Molecular Cloning: a Laboratory Manual. 2001. 3rd. ed.). Sequence comparisons can also be conducted using routine methods and include the use of commercial software programs such as Vector NTI (Invitrogen) and the free software package located at http://us.expasy.org.

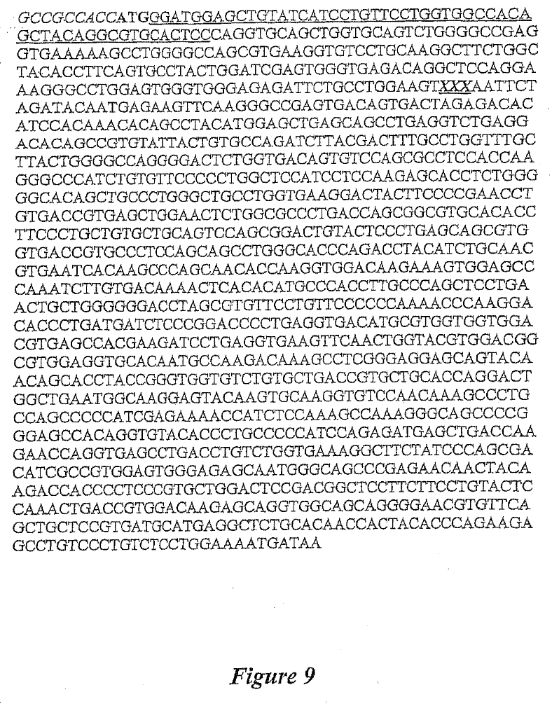

Preferably the nucleotide sequence is that of FIG. 9 wherein XXX is any codon encoding an amino acid residue other than Asparagine.

A third aspect of the invention provides an expression vector containing a nucleotide sequence encoding the modified antibody molecule according to the first aspect of the invention.

Preferably the nucleotide sequence is that of FIG. 9 wherein XXX is any codon encoding an amino acid residue other than Asparagine.

The DNA is then expressed in a suitable host to produce a polypeptide comprising the compound of the invention. Thus, the DNA encoding the polypeptide constituting the compound of the invention may be used in accordance with known techniques, appropriately modified in view of the teachings contained herein, to construct an expression vector, which is then used to transform an appropriate host cell such as a bacterium or eukaryotic cell for the expression and production of the polypeptide of the invention. Such techniques include those disclosed in U.S. Pat. Nos. 4,440,859 issued 3 Apr. 1984 to Rutter et al., 4,530,901 issued 23 Jul. 1985 to Weissman, 4,582,800 issued 15 Apr. 1986 to Crowl, 4,677,063 issued 30 Jun. 1987 to Mark et al. 4,678,751 issued 7 Jul. 1987 to Goeddel, 4,704,362 issued 3 Nov. 1987 to Itakura et al., 4,710,463 issued 1 Dec. 1987 to Murray, 4,757,006 issued 12 Jul. 1988 to Toole, Jr. et al., 4,766,075 issued 23 Aug. 1988 to Goeddel et al. and 4,810,648 issued 7 Mar. 1989 to Stalker, all of which are incorporated herein by reference.

Antibody expression conducted in eukaryotic host cells comprises nucleic acid encoding immunologically active antibody molecules, functionally linked to sequences capable of driving expression of said fragments in said host cell when said cell is cultured under conditions allowing said expression. The cells according to the invention are suitably used in large-scale production of antibody molecules.

Immortalized cells are known in the art, and can in principle grow indefinitely. Various tumor cell lines known in the art, including but not limited to cell lines such as Chinese hamster ovary (CHO) cell lines, HeLa, baby hamster kidney (BHK), hybridoma cell lines including NS0 and Sp2-0, are also immortalized. Cell lines commonly used for industrial manufacture of antibodies include CHO, NS0 and Sp2-0 (Chu and Robinson, 2001, Curr Opin Biotechnol 12, 180-187; Dempsey et al, 2003, Biotechnol Prog 19, 175-178; Yog et al. 2002, J. Immunol. Meth 261, 1-20).

Culturing of a eukaryotic cell is performed to enable it to metabolize, and/or grow and/or divide and/or produce recombinant proteins of interest. This can be accomplished by methods well known to persons skilled in the art, and includes but is not limited to providing nutrients for the cell. The methods comprise growth adhering to surfaces, growth in suspension, or combinations thereof.

Several culturing conditions can be optimized by methods well known in the art to optimize protein production yields. Culturing can be done for instance in dishes, roller bottles or in bioreactors, using batch, fed-batch, continuous systems, hollow fibre, and the like.

In order to achieve large scale (continuous) production of recombinant proteins through cell culture it is preferred in the art to have cells capable of growing in suspension, and it is preferred to have cells capable of being cultured in the absence of animal- or human-derived serum or animal- or human-derived serum components. Thus purification is easier and safety is enhanced due to the absence of additional, animal or human proteins derived from the culture medium, while the system is also very reliable as synthetic media are the best in reproducibility.

The conditions for growing or multiplying cells (see e.g. Tissue Culture, Academic Press, Kruse and Paterson, editors (1973)) and the conditions for expression of the recombinant product may differ somewhat, and optimization of the process is usually performed to increase the product yields and/or growth of the cells with respect to each other, according to methods generally known to the person skilled in the art. In general, principles, protocols, and practical techniques for maximizing the productivity of mammalian cell cultures can be found in Mammalian Cell Biotechnology: a Practical Approach (M. Butler, ed., IRL Press, 1991).

The antibody is expressed in the cells according to the invention, and may be recovered from the cells or preferably from the cell culture medium, by methods generally known to persons skilled in the art. Such methods may include precipitation, centrifugation, filtration, size-exclusion chromatography, affinity chromatography, cation- and/or anion-exchange chromatography, hydrophobic interaction chromatography, and the like.

The DNA encoding the polypeptide constituting the compound of the invention may be joined to a wide variety of other DNA sequences for introduction into an appropriate host. The companion DNA will depend upon the nature of the host, the manner of the introduction of the DNA into the host, and whether episomal maintenance or integration is desired.

Generally, the DNA is inserted into an expression vector, such as a plasmid, in proper orientation and correct reading frame for expression. If necessary, the DNA may be linked to the appropriate transcriptional and translational regulatory control nucleotide sequences recognised by the desired host, although such controls are generally available in the expression vector. Thus, the DNA insert may be operatively linked to an appropriate promoter. Bacterial promoters include the E. coli locI and lacZ promoters, the T3 and T7 promoters, the gpt promoter, the phage λ PR and PL promoters, the phoA promoter and the trp promoter. Eukaryotic promoters include the CMV immediate early promoter, the HSV thymidine kinase promoter, the early and late SV40 promoters and the promoters of retroviral LTRs. Other suitable promoters will be known to the skilled artisan. The expression constructs will desirably also contain sites for transcription initiation and termination, and in the transcribed region, a ribosome-binding site for translation (Hastings et al., International Patent No. WO 98/16643, published 23 Apr. 1998)

The vector is then introduced into the host through standard techniques, such as for instance transfection, lipofection, electroporation, virus infection and the like (see Sambrook, J. and Russell, D. W. (2001). Molecular cloning: a laboratory manual, Pub Cold Spring Harbor Laboratory Press).

The introduced nucleic acid can be present in the cells extrachromosomally, whereupon expression is transient. Extrachromosomal plasmids may or may not be able to divide in the host cell; when plasmids are not able to replicate the plasmid becomes diluted in the cells as they divide and hence expression is lost in time. If plasmids are stably integrated in the genome of said cells, cells can drive expression in a stable manner; this is the preferred case for industrial antibody production.

The DNA encoding the light chain and the DNA encoding the heavy chain of any antibody may be present on the same plasmid or on separate plasmids. Coding regions are inserted into the expression plasmid adjacent to the promoter by methods generally known to persons skilled in the art (see Sambrook, J. and Russell D. W. (2001), Molecular cloning: a laboratory manual. Pub. Cold Spring Harbor Laboratory Press). Coding regions may also include leader or signal peptides which facilitate secretion of the protein into the medium: this aids recovery and purification of said protein. Such signal peptides are cleaved from the precursor polypeptides upon secretion to give the mature polypeptides and hence antibody.

Generally, not all of the hosts will be transformed by the vector and it will therefore be necessary to select for transformed host cells. One selection technique involves incorporating into the expression vector a DNA sequence marker, with any necessary control elements, that codes for a selectable trait in the transformed cell. These markers include dihydrofolate reductase, G418 or neomycin resistance for eukaryotic cell culture, and tetracycline, kanamycin or ampicillin resistance genes for culturing in E. coli and other bacteria. Alternatively, the gene for such selectable trait can be on another vector, which is used to CD-transform the desired host cell. The expression vector may also encode an enzyme, for example glutamine synthetase, for which the parent cell is deficient. Only cells in which the vector has stably incorporated in the genome are able to grow in a medium lacking the essential nutrient produced by the enzyme, in the case of glutamine synthetase a glutamine-free medium.

Host cells that have been transformed by the recombinant DNA of the invention are then cultured for a sufficient time and under appropriate conditions known to those skilled in the art in view of the teachings disclosed herein to permit the expression of the polypeptide, which can then be recovered.

The polypeptide of the invention can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxylapatite chromatography, size exclusion chromatography, and lectin chromatography.

Many expression systems are known, including systems employing: bacteria (e.g. E. coli and Bacillus subtilis) transformed with, for example, recombinant bacteriophage, plasmid or cosmid DNA expression vectors; yeasts (e.g. Saccharomyces cerevisiae) transformed with, for example, yeast expression vectors; insect cell systems transfected with, for example, viral expression vectors (e.g. baculovirus); plant cell systems transfected with, for example, viral or bacterial expression vectors; animal cell systems transfected with, for example, adenovirus expression vectors.

The vectors can include a prokaryotic replicon, such as the Col E1 ori, fox propagation in a prokaryote, even if the vector is to be used for expression in other, non-prokaryotic cell types. The vectors can also include an appropriate promoter such as a prokaryotic promoter capable of directing the expression (transcription and translation) of the genes in a bacterial host cell, such as E. coli, transformed therewith.

A promoter is an expression control element formed by a DNA sequence that permits binding of RNA polymerase and transcription to occur. Promoter sequences compatible with exemplary bacterial hosts are typically provided in plasmid vectors containing convenient restriction sites for insertion of a DNA segment of the present invention.

Typical prokaryotic vector plasmids are: pUC18, pUC19, pBR322 and pBR329 available from Biorad Laboratories (Richmond, Calif., USA); pTrc99A, pKK223-3, pKK233-3, pDR540 and pRIT5 available from Pharmacia (Piscataway, N.J., USA); pBS vectors, Phagescript vectors, Bluescript vectors, pNH8A, pNH16A, pNH18A, pNH46A available from Stratagene Cloning Systems (La Jolla, Calif. 92037, USA).

Some well-known and much used promoters for expression in eukaryotic cells comprise promoters derived from viruses, such as adenovirus, e.g. the E1A promoter, promoters derived from cytomegalovirus (CMV), such as the CMV immediate early QE) promoter, promoters derived from Simian Virus 40 (SV40), and the like. Suitable promoters can also be derived from eukaryotic cells, such as metallothionein (MT) promoters, elongation factor 1α (EF-1α) promoter, actin promoter, an immunoglobulin promoter, heat shock promoters, and the like.

A typical mammalian cell vector plasmid is pSVL available from Pharmacia (Piscataway, N.J., USA). This vector uses the SV40 late promoter to drive expression of cloned genes, the highest level of expression being found in T antigen-producing cells, such as COS-1 cells. An example of an inducible mammalian expression vector is pMSG, also available from Pharmacia (Piscataway, N.J., USA). This vector uses the glucocorticoid-inducible promoter of the mouse mammary-tumour virus long terminal repeat to drive expression of the cloned gene. A further, example of a eukaryotic expression vector is pEE series (Lonza) which uses a human cytomegalovirus (CMV) major immediate early promoter to control expression.

Useful yeast plasmid vectors are pRS403-406 and pRS413-416 and are generally available from Stratagene Cloning Systems (La Jolla, Calif. 92037, USA). Plasmids pRS403, pRS404, pRS405 and pRS406 are Yeast integrating plasmids (YIps) and incorporate the yeast selectable markers HIS3, TRP1, LEU2 and URA3. Plasmids pRS413-416 are Yeast Centromere plasmids (YCps).

Methods well known to those skilled in the art can be used to construct expression vectors containing the coding sequence and, for example appropriate transcriptional or translational controls. Such methods are described in Sambrook, J. and Russell, D. W. (2001), Molecular cloning: a laboratory manual. Pub. Cold Spring Harbor Laboratory Press.

One such method involves ligation via homopolymer tails. Homopolymer polydA (or polydC) tails are added to exposed 3′ OH groups on the DNA fragment to be cloned by terminal deoxynucleotidyl transferases. The fragment is then capable of annealing to the polydT (or polydG) tails added to the ends of a linearised plasmid vector. Gaps left following annealing can be filled by DNA polymerase and the free ends joined by DNA ligase.

Another method involves ligation via cohesive ends. Compatible cohesive ends can be generated on the DNA fragment and vector by the action of suitable restriction enzymes. These ends will rapidly anneal through complementary base pairing and remaining nicks can be closed by the action of DNA ligase.

A further method uses synthetic molecules called linkers and adaptors. DNA fragments with blunt ends are generated by bacteriophage T4 DNA polymerase or E. coli DNA polymerase I which remove protruding 3′ termini and fill in recessed 3′ ends. Synthetic linkers, pieces of blunt-ended double-stranded DNA which contain recognition sequences for defined restriction enzymes, can be ligated to blunt-ended DNA fragments by T4 DNA ligase. They are subsequently digested with appropriate restriction enzymes to create cohesive ends and ligated to an expression vector with compatible termini. Adaptors are also chemically synthesised DNA fragments which contain one blunt end used for ligation but which also possess one pre-formed cohesive end.

Synthetic linkers containing a variety of restriction endonuclease sites are commercially available from a number of sources including International Biotechnologies Inc (Kodak/IBI), New Haven, Conn., USA.

An alternative method for the construction of expression vectors includes the use of PCR technology (see Sambrook, J. and Russell, D. W. (2001). Molecular cloning: a laboratory manual. Pub. Cold Spring Harbor Laboratory Press)

Thus, in a fourth aspect of the invention there is provided a host cell producing a modified antibody molecule according to the first aspect of the invention, resulting from the expression of the nucleotide sequence encoding the modified antibody molecule.

Preferably the nucleotide sequence is that of FIG. 9 wherein XXX is any codon encoding an amino acid residue.

In a fifth aspect, the invention provides the modified antibody molecule according to the first aspect of the invention in association with at least one other agent.

Preferably the agent is at least one selected from drugs, toxins (e.g. PE, DT, Ricin A etc.), radionuclides (e.g. ⁹⁰Y, ¹³¹I, ¹²⁵I, ^{99m}Tc etc.), nucleases (e.g. RNases, caspases and DNases), enzymes, cytokines (e.g. IL-12) and chemokines.

Examples of the use of antibodies in association with otter agents is described in GB 2 360 772 and GB 2 383 538.

More preferably the agent is conjugated to the modified antibody molecule, such a conjugate includes a fusion protein.

Most preferably the agent is a cytokine.

A sixth aspect of the invention provides a composition comprising the modified antibody molecule according to the first aspect of the invention and a pharmaceutically acceptable carrier, excipient and/or diluent.

Preferably the composition comprises the nucleotide sequence as described in the second aspect of the invention.

More preferably the composition further comprises at least one other agent.

Most preferably the agent is at least one selected from drags, toxins (e.g. PE, DT, Ricin A etc.), radionuclides (e.g. ⁹⁰Y ¹³¹I, ¹²⁵I, ^{99m}Tc etc.), nucleases (e.g. RNases, DNases), proteases (e.g. caspases), enzymes for prodrug approaches, cytokines, chemokines.

In a seventh aspect of the invention there is provided the use of a modified antibody molecule according to the first aspect of the invention in the manufacture of a medicament for the treatment and/or diagnosis and/or prevention of Cancer, inflammatory disorders such as Crohn's disease, rheumatoid arthritis, psoriasis, asthma and allergies; cardiovascular diseases such as restenosis, cardiopulmonary bypass, myocardial infarction; infectious diseases such as respiratory syncytial virus, HIV, hepatitis C; autoimmune disorders such as lupus and dermatomyositis; Central Nervous System disorders such as Alzheimer's, transplant rejection and graft-versus-host disease; and nephritis, sepsis, hemoglobinuria, chemotherapy induced thrombocytopenia, and addiction (e.g. cocaine and nicotine).

Preferably the disease is Cancer. More preferably the cancer is a cancer of the Breast, Ovary, Uterus, Lung, Prostate, Colon, B-NHL, multiple myeloma, AML, CLL and hairy cell leukaemia.

In an eighth aspect of the invention there is provided a phage containing the nucleotide acid according to the second aspect of the invention.

Preferably the nucleotide sequence is operably linked to the nucleotide sequence encoding a phage surface protein. More preferably the nucleotide sequence is expressed by the phage and displayed on the phage surface.

The invention further provides the use of the phage described above in a screening assay.

Such a screening assay would identify mutant antibodies, antibody fragments or antibody derivatives displayed on phage that were able to bind a selective target. Preferably that target is the MUC-1 gene product.

The display of proteins and polypeptides on the surface of bacteriophage (phage), fused to one of the phage coat proteins, provides a powerful tool for the selection of selective ligands. This ‘phage display’ technique was originally used by Smith in 1985 (Science 228, 1315-7) to create large libraries of peptides for the purpose of selecting those, with high affinity for a particular antigen. More recently, the method has been employed to present antibodies at the surface of phage in order to identify ligands having desired properties (McCafferty et al., Nature, 1990, 552-554).

The use of phage display to isolate ligands that bind biologically relevant molecules has been reviewed in Felici et al. (1995) Biotechnol Annual Rev. 1, 149-183, Katz (1997) Annual Rev. Biophys. Biomol. Struct. 26, 27-45 and Hoogenboom et al. (1998) Immunotechnology 4(1), 1-20. Several randomised combinatorial peptide libraries have been constructed to select for polypeptides that bind different targets, e.g. cell surface receptors or DNA (reviewed by Kay, 1995, Perspect. Drug Discovery Des. 2, 251-268; Kay and Paul, 1996, Mol. Divers. 1, 139-140). Proteins and multimeric proteins have been successfully phage-displayed as functional molecules (see EP 0349578A, EP 0527839A, EP 0589S77A; Chiswell and McCafferty, 1992, Trends Biotechnol 10, 80-84). In addition, functional antibody fragments (e.g. Fab, single chain Fv [scFv]) have been expressed (McCafferty et al., 1990, Nature 348, 552-554; Barbas et al., 1991, Proc. Natl. Acad. Sci. USA 88, 7978-7982; Clackson et al., 1991, Nature 352, 624-628), and some of the shortcomings of human monoclonal antibody technology have been superseded since human high affinity antibody fragments have been isolated (Marks et al., 1991, J. Mol. Biol 222, 581-597; Hoogenboom and Winter, 1992, J. Mol. Biol 227, 381-388). Further information on the principles and practice of phage display is provided in Phage display of peptides and proteins: a laboratory manual Ed Kay, Winter and McCafferty (1996) Academic Press, Inc ISBN 0-12-402380-0, the disclosure of which is incorporated herein by reference.

The display of mutant antibody fragments on phage provides methods to detect selective binding of a polypeptide e.g. MUC-1 gene product to the test ScFV being expressed

In an ninth aspect of the invention there is provided a method of modifying an antibody molecule to produce a modified antibody by substitution of at least one amino acid residue that forms part of a glycosylation site in the variable region of an unmodified parent antibody molecule with a different amino acid residue, characterised in that the resulting modified antibody is not glycosylated at the previous glycosylation site of which the amino acid modification forms part and the resulting modified antibody exhibits a greater binding affinity for the specific target than the unmodified parent antibody molecule.

Methods of modifying antibody molecules will be known by those skilled in the art such that the antibody molecule of the invention may be modified by known polypeptide modification techniques.

### Meanings of Terms Used

The term “antibody molecule” shall be taken to refer to any one of an antibody, an antibody fragment, or antibody derivative. It is intended to embrace wildtype IgG antibodies (i.e. a molecule comprising four polypeptide chains), other intact antibodies (e.g. IgA, IgM, camelid antibodies), synthetic antibodies, recombinant antibodies or antibody hybrids, such as, but not limited to, a single-chain modified antibody molecule produced by phage-display of immunoglobulin light and/or heavy chain variable and/or constant regions, or other immunointeractive molecule capable of binding to an antigen in an immunoassay format that is known to those skilled in the art.

The term “antibody derivative” refers to any modified antibody molecule that is capable of binding to an antigen in an immunoassay format that is known to those skilled in the art, such as a fragment of an antibody (e.g. Fab or Fv fragment), or a modified antibody molecule that is modified by the addition of one or more amino acids or other molecules to facilitate coupling the antibodies to another peptide or polypeptide, to a large carrier protein or to a solid support (e.g. the amino acids tyrosine, lysine, glutamic acid, aspartic acid, cysteine and derivatives thereof, NR₂-acetyl groups or COOH-terminal amido groups, amongst others).

The term “ScFv molecule” refers to any molecules wherein the V_{H} and V_{L} partner domains are linked via a flexible oligopeptide.

The term “diabody” refers to any molecules that are non-covalently associated dimers, in which each chain comprises two domains consisting of V_{H} and V_{L} domains. Both domains are connected by a linker that is too short to allow pairing between domains of the same chain. Thus, each chain alone is not capable of binding antigen, but two chains will assemble to dimeric diabodies with two functional antigen binding sites.

The terms “nucleotide sequence” or “nucleic acid” or “polynucleotide” or “oligonucleotide” are used interchangeably and refer to a heteropolymer of nucleotides or the sequence of these nucleotides. These phrases also refer to DNA or RNA of genomic or synthetic origin which may be single-stranded or double-stranded and may represent the sense or the antisense strand, to peptide nucleic acid (PNA) or to any DNA-like or RNA-like material. In the sequences herein A is adenine, C is cytosine, T is thymine, G is guanine and N is A, C, G or T (U). It is contemplated that where the polynucleotide is RNA, the T (thymine) in the sequences provided herein is substituted with U (uracil). Generally, nucleic acid segments provided by this invention may be assembled from fragments of the genome and short oligonucleotide linkers, or from a series of oligonucleotides, or from individual nucleotides, to provide a synthetic nucleic acid which is capable of being expressed in a recombinant transcriptional unit comprising regulatory elements derived from a microbial or viral operon, or a eukaryotic gene.

The terms “probe” and “primer” are used interchangeably and refer to a sequence of nucleotide residues which are at least about 5 nucleotides, more preferably at least about 7 nucleotides, more preferably at least about 9 nucleotides, more preferably at least about 11 nucleotides and most preferably at least about 17 nucleotides. The probe is preferably less than about 500 nucleotides, preferably less than about 200 nucleotides, more preferably less than about 100 nucleotides, more preferably less than about 50 nucleotides and most preferably less than 30 nucleotides. Preferably the probe is from about 6 nucleotides to about 200 nucleotides, preferably from about 15 to about 50 nucleotides, more preferably from about 17 to 30 nucleotides and most preferably from about 20 to 25 nucleotides. Preferably the fragments can be used in polymerase chain reaction (PCR), various hybridisation procedures or microarray procedures to identify or amplify identical or related parts of mRNA or DNA molecules. A fragment or segment may uniquely identify each polynucleotide sequence of the present invention.

The terms “polypeptide” or “peptide” or “amino acid sequence” refer to an oligopeptide, peptide, polypeptide or protein sequence or fragment thereof and to naturally occurring or synthetic molecules. A polypeptide “fragment,” “portion,” or “segment” is a stretch of amino acid residues of at least 2 residues, preferably about 5 amino acids, preferably at least about 7 amino acids, more preferably at least about 9 amino acids and most preferably at least about 17 or more amino acids. To be active, any polypeptide must have sufficient length to display biological and/or immunological activity.

The term “variant” (or “analogue”) refers to any polypeptide differing from naturally occurring polypeptides by amino acid insertions, deletions, and substitutions, created using, e.g., recombinant DNA techniques. Guidance in determining which amino acid residues may be replaced, added or deleted without abolishing activities of interest, may be found by comparing the sequence of the particular polypeptide with that of homologous peptides and minimising the number of amino acid sequence changes made in regions of high homology (conserved regions) or by replacing amino acids with consensus sequence.

The terms “purified” or “substantially purified” as used herein denotes that the indicated nucleic acid or polypeptide is present in the substantial absence of other biological macromolecules, e.g., polynucleotides, proteins, and the like. In one embodiment the polynucleotide or polypeptide is purified such that it constitutes at least 95% by weight, more preferably at least 99% by weight, of the indicated biological macromolecules present (but water, buffers, and other small molecules, especially molecules having a molecular weight of less than 1000 daltons, can be present).

The term “isolated” as used herein refers to a nucleic acid or polypeptide separated from at least one other component (e.g., nucleic acid or polypeptide) present with the nucleic acid or polypeptide in its natural source. In one embodiment, the nucleic acid or polypeptide is found in the presence of (if anything) only a solvent, buffer, ion, or other component normally present in a solution of the same. The terms “isolated” and “purified” do not encompass nucleic acids or polypeptides present in their natural source.

The term “recombinant,” when used herein to refer to a polypeptide or protein, means that a polypeptide or protein is derived from recombinant (e.g., microbial, insect, or mammalian) expression systems. “Microbial” refers to recombinant polypeptides or proteins made in bacterial or fungal (e.g., yeast) expression systems. As a product, “recombinant microbial” defines a polypeptide or protein essentially free of native endogenous substances and unaccompanied by associated native glycosylation. Polypeptides or proteins expressed in most bacterial cultures, e.g., E. coli, will be free of glycosylation modifications; polypeptides or proteins expressed in yeast will have a glycosylation pattern in general different from those expressed in mammalian cells.

The term “expression vector” refers to a plasmid or phage or virus or vector, for expressing a polypeptide from a DNA (RNA) sequence. An expression vehicle can comprise a transcriptional unit comprising an assembly of (1) a genetic element or elements having a regulatory role in gene expression, for example, promoters and often enhancers, (2) a structural or coding sequence which is transcribed into mRNA and translated into protein, and (3) appropriate transcription and translation initiation and termination sequences. Structural units intended for use in yeast or eukaryotic expression systems preferably include a leader sequence enabling extracellular secretion of translated protein by a host cell. Alternatively, where recombinant protein is expressed without a leader or transport sequence, it may include an amino terminal methionine residue. This residue may or may not be subsequently cleaved from the expressed recombinant protein to provide a final product.

The terms “selective binding” and “binding selectivity” indicates that the variable regions of the antibodies of the invention recognise and bind, polypeptides of the invention exclusively (i.e., able to distinguish the polypeptide of the invention from other similar polypeptides despite sequence identity, homology, or similarity found in the family of polypeptides), but may also interact with other proteins (for example, S. aureus protein A or other antibodies in ELISA techniques) through . . . interactions with sequences outside the variable region of the antibodies, and in particular, in the constant region of the molecule. Screening assays to determine binding selectivity of an antibody of the invention are well known and routinely practiced in the art. For a comprehensive discussion of such assays, see Harlow et al. (Eds), Antibodies A Laboratory Manual; Cold Spring Harbor Laboratory; Cold Spring Harbor, N.Y., (1988), Chapter 6. Antibodies that recognise and bind fragments of the polypeptides of the invention are also contemplated, provided that the antibodies are first and foremost selective for, as defined above, full-length polypeptides of the invention. As with antibodies that are selective for full length polypeptides of the invention, antibodies of the invention that recognise fragments are those which can distinguish polypeptides from the same family of polypeptides despite inherent sequence identity, homology, or similarity found in the family of proteins.

The term “binding affinity” includes the meaning of the strength of binding between an antibody molecule and an antigen.

### PREFERRED EMBODIMENTS

Examples embodying certain preferred aspects of the invention will now be described with reference to the following figures in which:—

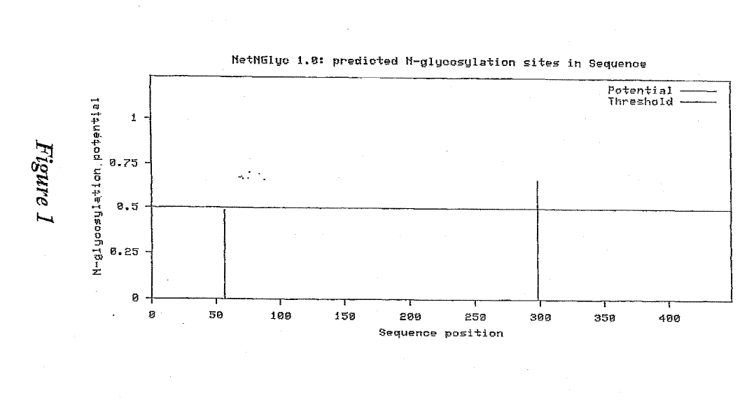

FIG. 1—Prediction of N-glycosylation by programme NetNGlyc.

While the probability that N56 is glycosylated is below the threshold, it is very close to this value. This appears to explain why the N56 position is not glycosylated in every molecule thereby producing the different glycoforms.

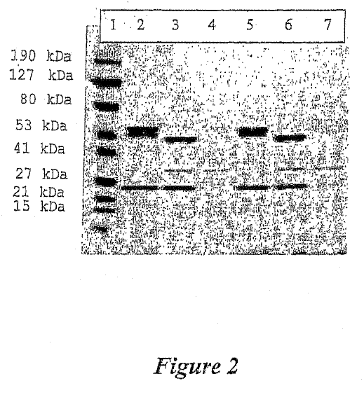

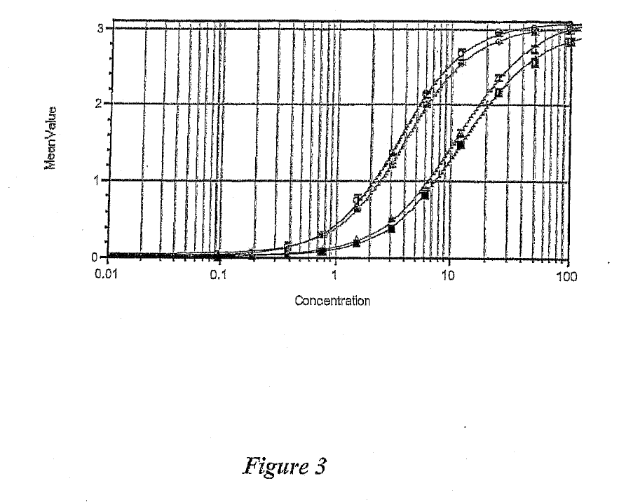

FIG. 2—SDS-PAGE gel of HMFG1 stained with Coomassie Blue.

Lane 1: Molecular weight markers

Lane 2: Sample 1 HMFG1

Lane 3: Sample 1 HMFG1 treated with PNGaseF

Lane 4: PNGaseF only

Lane 5: Sample 2HMFG1

Lane 6: Sample 2 HMFG1 treated with PNGaseF

Lane 7: PNGaseF only

Treatment with PNGaseF (New England Biolabs) was performed according to manufacturer's instructions, PNGaseF (peptide N-glycosidase F) is visible on the gel when present (as seen in lanes 4 and 7). SDS-Page (sodium dodecyl sulphate polyacrylamide gel electrophoresis) gels and related reagents were from Invitrogen, and used according to the manufacturers instructions.

FIG. 3—Antigen binding ELISA.

Two samples were analysed.

Sample 1: squares=untreated, circles=PNGaseF treated

Sample 2: triangles untreated, diamonds=PNGaseF treated

The units of the two axes are: x-axis-|μg/mL y-axis-optical density.

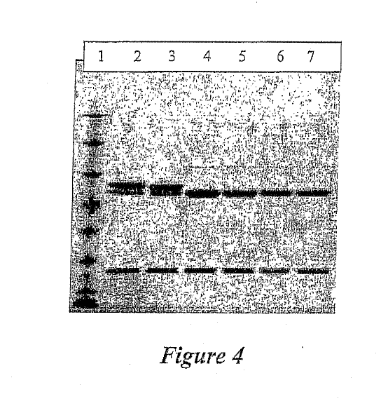

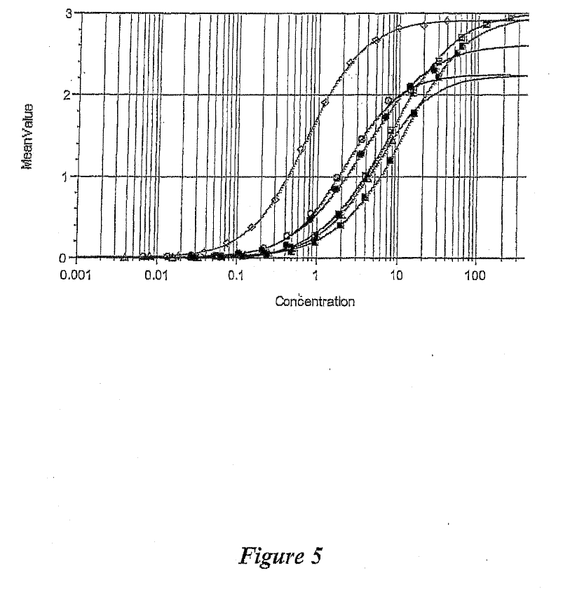

FIG. 4—Reducing SDS-PAGE gel of four of the HMFG1 mutants stained with Coomassie Blue.

Lane 1: Molecular weight markers

Lane 2: HMFG1 standard

Lane 3: WT HMFG1

Lane 4: N56S HMFG1

Lane 5: N56G HMFG1

Lane 6: N56Q HMFG1

Lane 7: N56D HMFG1

Lane 2 is standard GMP material; lanes 3 to 7 are purified proteins from transient transfections.

FIG. 5—Antigen binding ELSA with mutant HMFG1

| | |
|---|---|
| HMFG1 standard: | open squares |
| Wildtype (WT) HMFG1: | filled squares |
| N56G HMFG1: | open diamonds |
| N56S HMPG1 | filled circles |
| N56Q HMFG1 | open triangles |
| N56D HMFG1 | open circles |

The x-axis is in μg/ml and the y-axis is optical density.

The ELISA (enzyme linked immunosorbent assay) curve is shifted to the left, indicating a higher affinity for antigen.

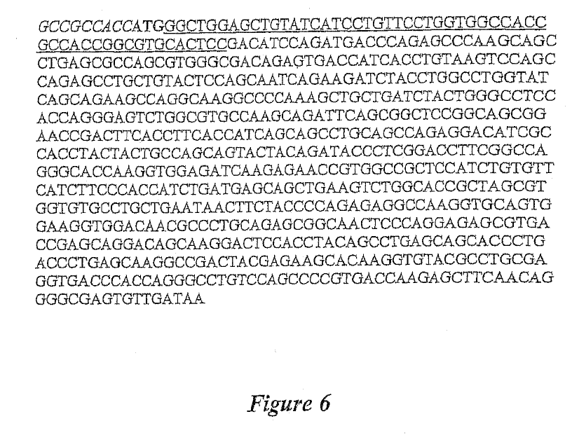

FIG. 6—Wild-type light chain DNA sequence with Kozak in italics, start codon in bold and leader sequence underlined

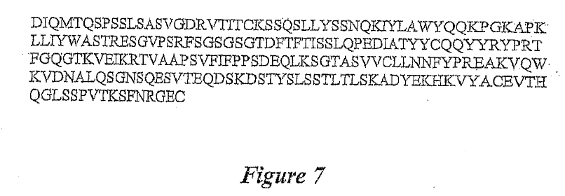

FIG. 7—Sequence of secreted light chain

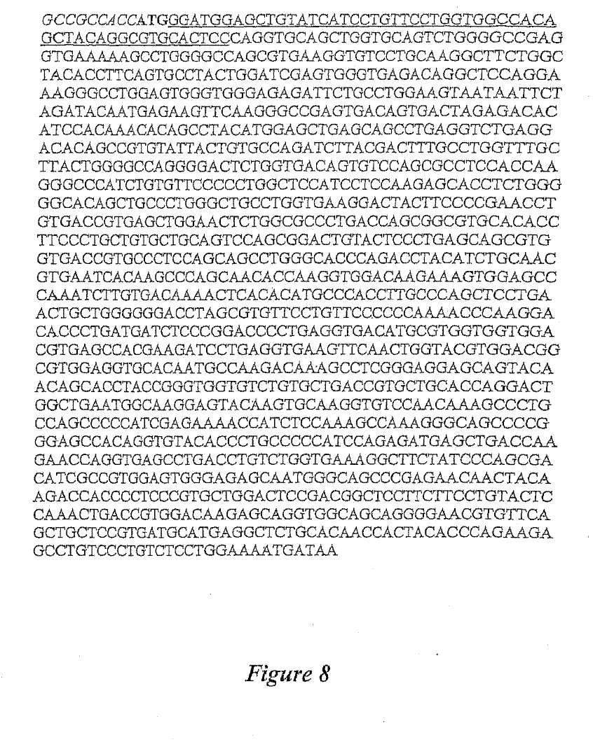

FIG. 8—Wild-type heavy chain DNA sequence with Kozak in italics, start codon in bold and leader sequence underlined

FIG. 9—Generic heavy chain DNA sequence with Kozak in italics, start codon in bold and leader sequence underlined. Codon for modification is marked as XXX.

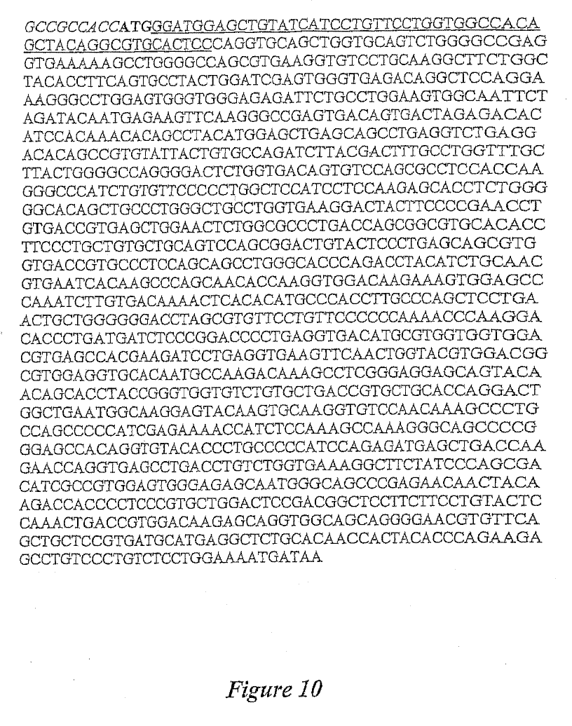

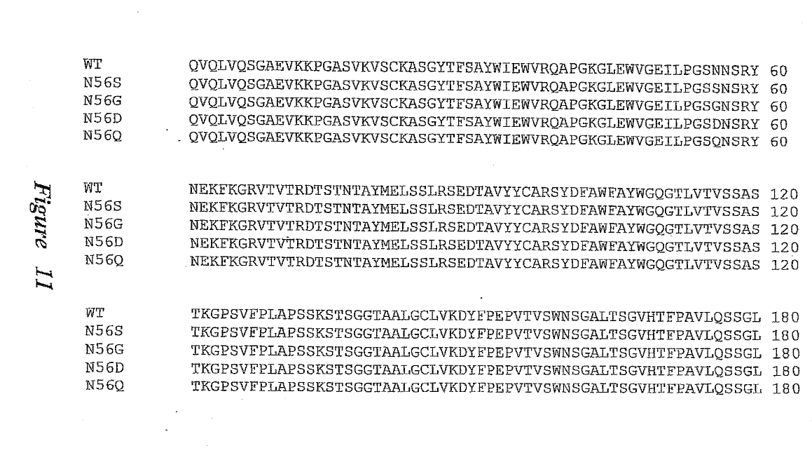

FIG. 10—N56G heavy chain DNA sequence with Kozak in italics, start codon in bold and leader sequence underlined

FIG. 11—Alignment of amino acid sequences of secreted heavy chains.

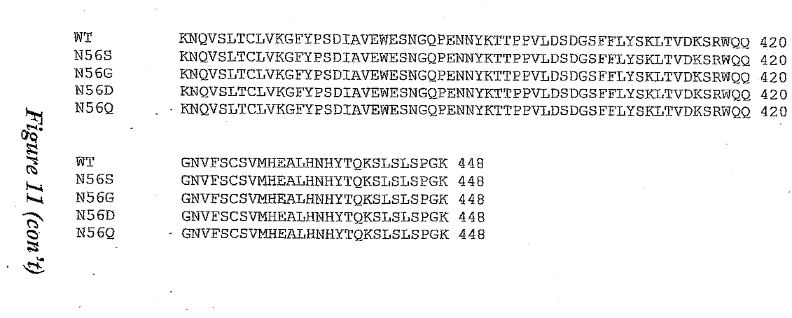

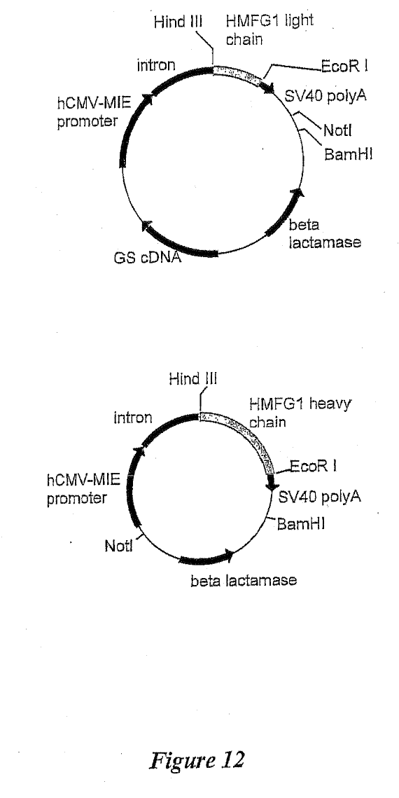

FIG. 12—Vector maps for the separate production of HMFG1 heavy and light chains.

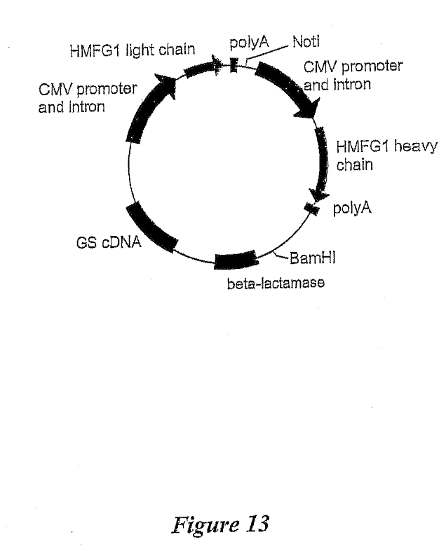

FIG. 13. Plasmid map of single vector for production of HMFG1.

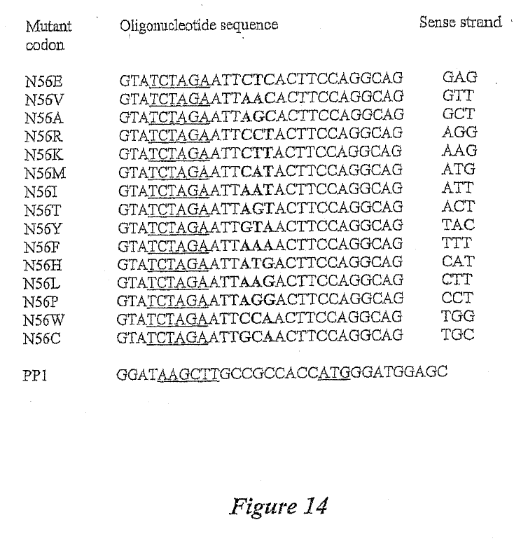

FIG. 14. Oligonucleotide sequences used for PCR mutagenesis. XbaI restriction site is underlined; mutated codon is in bold. The mutated codon is repeated in the final column as a sense strand codon coding for the requisite amino acid. In PP1, HindIII and the ATG start codon are underlined.

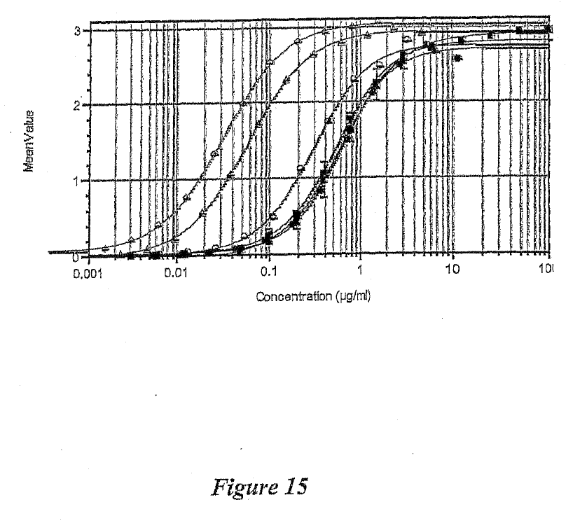

FIG. 15. Antigen binding ELISA with mutant HMFG1 from stable cell lines (part 1).

| | |
|---|---|
| HMFG1 standard: | filled squares |
| N56N: | open squares |
| N56K: | open circles |
| N56G: | open triangles |
| N56C: | open diamonds |
| N56Q: | filled circles |

The x-axis is in μg/ml and the y-axis is optical density.

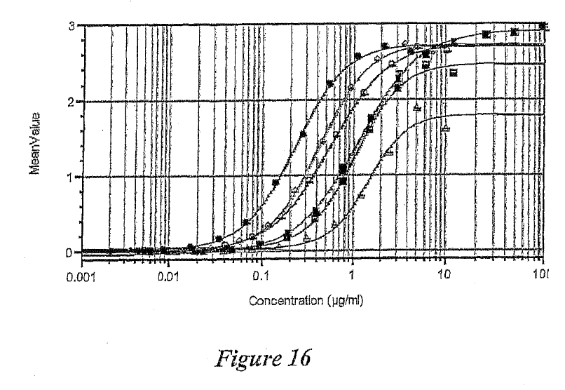

FIG. 16. Antigen binding ELISA with mutant HMFG1 from stable cell lines (part 2).

| | |
|---|---|
| HMFG1 standard: | filled squares |
| N56P: | open squares |
| N56L: | open circles |
| N56I: | open triangles |
| N56D: | open diamonds |
| N56A: | filled circles |

The x-axis is in μg/ml and the y-axis is optical density.

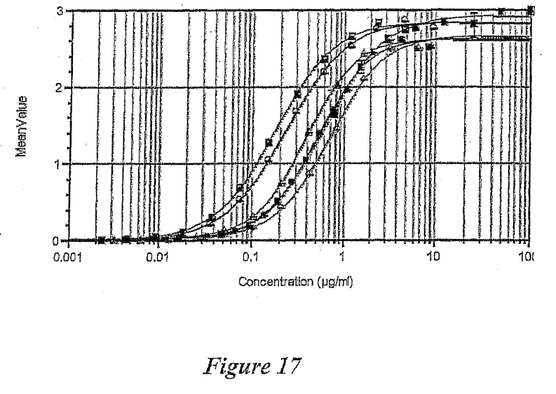

FIG. 17. Antigen binding ELISA with mutant HMFG1 from stable cell lines (part 3).

| | |
|---|---|
| HMFG1 standard: | filled squares |
| N56M: | open squares |
| N56R: | open circles |
| N56W: | open triangles |
| N56H: | open diamonds |
| N56F: | filled circles |

The x-axis is in μg/ml and the y-axis is optical density.

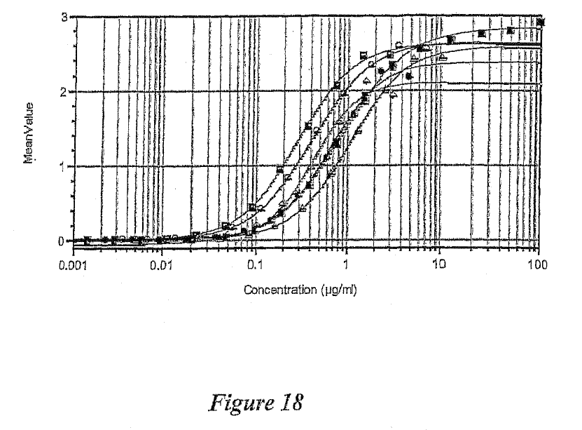

FIG. 18. Antigen binding ELISA with mutant HMFG1 from stable cell lines (part 4).

| | |
|---|---|
| HMFG1 standard: | filled squares |
| N56S: | open squares |
| N56Y: | open circles |
| pAS6K: | open triangles |
| N56T: | open diamonds |
| N56E: | filled circles |

The x-axis is in μg/ml and the y-axis is optical density.

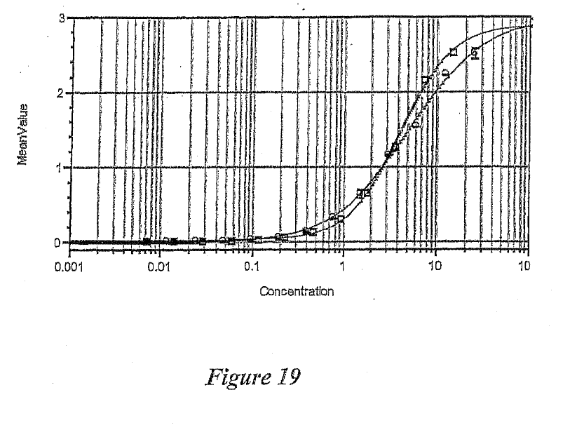

FIG. 19. Antigen binding ELISA with mutant N56V from transient transfection.

| | |
|---|---|
| HMFG1 standard: | open circles |
| N56V: | open squares |

The x-axis is in μg/ml and the y-axis is optical density.

This mutant shows similar affinity to antigen as HMFG1.

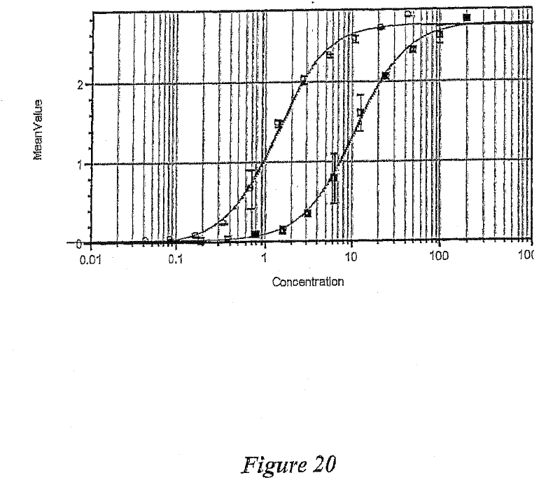

FIG. 20. Antigen binding ELISA with mutant N56G from NS0 stable transfection.

| | |
|---|---|
| HMPG1 standard: | open squares |
| N56G: | open circles |

The x-axis is in μg/ml and the y-axis is optical density.

This mutant again shows a shift to the left, indicating higher a affinity to antigen than HMFG1.

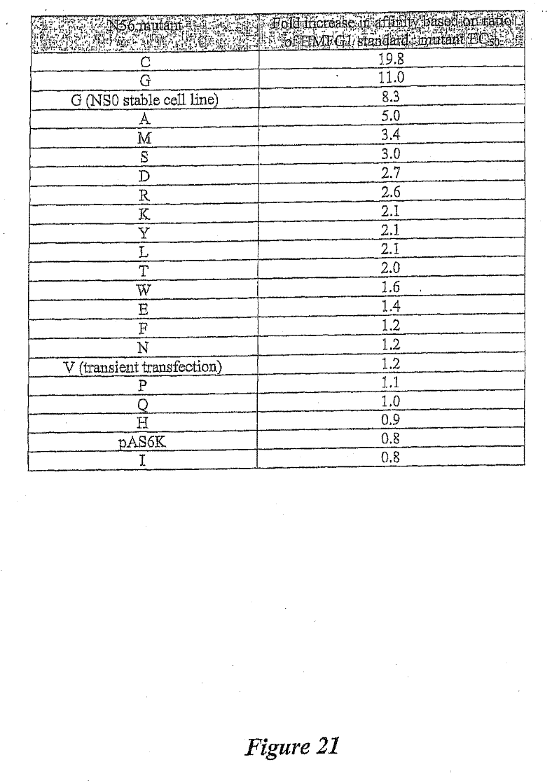

FIG. 21. Summary table of data shown in FIGS. 15-20.

EC₅₀ values were calculated from the ELISA curves, using ELISA software package SoftMax Pro. Values were compared to the EC₅₀ obtained for the HMFG1 standard used in the same assay in all cases.

Two values are shown for N56G: the difference in these values indicates the intrinsic variation in these assays.

Values are also shown for N56N (codon optimised HMFG1 DNA sequence) and pAS6K (non-codon-optimised HMFG1 DNA sequence); again this difference reflects assay variation.

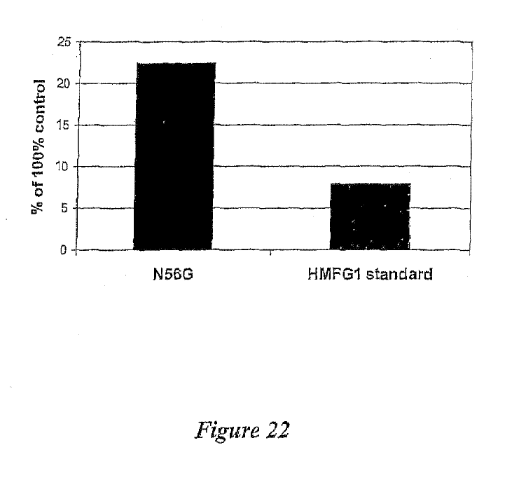

FIG. 22 Antibody dependent cell mediated cytotoxicity (ADCC) test.

The ADCC results shows that the N56G was more potent at mediating ADCC than HMFG1 standard, most likely due to its increased affinity. This effect was seen at a range of antibody concentrations.

### Example 1

### Glycosylation in Variable Region of HMFG1 Heavy Chain

The wildtype sequence of the HMFG1 antibody was analysed for potential N-glycosylation sites in silico using the NetNGlyc program (http://www.cbs.dtu.dk/services/NetNGlyc/ (Technical University of Denmark) or equivalent software programs). The NetNGlyc software trains artificial neural networks on the surrounding sequence context around a potential glycosylation site, in an attempt to discriminate between acceptor and non-acceptor sequons.

This analysis identified two asparagine residues in the heavy chain that were potential sites of glycosylation (FIG. 1). The second site is in the constant region and is the asparagine on which IgG molecules are expected to be glycosylated. (Asn298 (also referred to as Asn297 under the KABAT numbering system), a conserved N-glycosylated residue present in normal immunoglobulin G molecules) However the other site was Asn56 in the variable (antigen-binding, CDR2) region.

When HMFG1 is electrophoresed on reducing SDS-PAGE and stained with Coomassie Blue, the heavy chain appears as a doublet (FIG. 2). However when this is treated with PNGaseF (an endoglycosidase which removes all N-glycans from proteins), both bands decrease in molecular weight to the same size. This indicates that the two bands are alternate glycoforms of the same protein.

Antibodies are normally glycosylated in the constant region, but these IgG glycans are limited in structure. A typical Fc-region glycan comprises the basic core structure of two N-acetylglucosamines, three mannoses and two further N-acetylglucosamines on each antenna.

This basic structure usually contains a core fucose sugar, and may contain a bisecting N-acetylglucosamine, and antennae terminating with two, one or no galactose residues, (see Jefferis et al. (1998) Immunological Reviews 163 p 59-76 and Raju et al. (2000) Glycobiology 10 p 477-486).

The Fc glycan does not cause the heavy chain to electrophorese as two bands on SDS-PAGE, and the presence of a doublet indicates partial glycosylation elsewhere on the protein, likely at the site indicated from computer analysis.

Wheal samples of native HMFG1 and PNGaseF-treated (i.e. deglycosylated) HMFG1 are tested in a MUC1 antigen-binding ELISA (see Example 3), the deglycosylated samples appear to have an increased affinity for MUC1 (FIG. 3) which further suggests that there is glycosylation in the antigen-binding region.

### Example 2

### Construction of Expression Vectors and Transfection

In order to generate mutations of Asn56, it was decided to synthesise the DNA encoding HMFG1 to concomitantly optimise codon usage, while also generating mutant forms of the gene.

Codon optimisation and DNA synthesis was performed by GeneArt GMBH (Germany) www.geneart.com/gene-synthesis. Codon optimised genes are commonly used to increase expression of the gene. Typically only common codons are included and sequences that may result in secondary structure formation in the mRNA are altered. There is no change to the polypeptide sequence and changes will have no impact on the protein produced. DNA sequence encoding the wildtype HMFG1 amino acid sequence and also four mutated heavy chains were synthesised in this way. These mutants were N56S, N56G, N56D and N56Q.

The following sequences derived from the synthesis are shown in the figures and include:
Wild-type light chain
Wild-type heavy chain
Generic N56XXX substitution heavy chain
N56G heavy chain (as ah example)
Alignment of four mutant heavy chain amino acid sequence with Asn wild-type sequence.

DNA sequences were supplied in a standard cloning vector, so sequences were amplified using PCR and inserted into suitable expression vectors.

One method of synthesising modified antibody that was applied was the light chain DNA was cloned into pEE12.1 vector and the heavy chain DNA into pEE6.4 vector as EcoRI-HindIII fragments (both vectors are from Lonza Biologics) (see FIG. 12).

Expression cassettes included a Kozak sequence and a leader sequence (see FIGS. 8 to 10). The Kozak sequence is contiguous with the ATG start codon and enables the ribosome to recognise the ATG start of translation, thereby improving expression. The sequence of inserts in these plasmids was confirmed by DNA sequencing (DBS Genomics). Transcription is from a CMV promoter.

Plasmids were transiently transfected into CHO cells using Lipofectamine 2000 (Invitrogen) according to manufacturer's instructions and the medium harvested so after 3 to 4 days. Plasmids encoding the light and heavy chain were co-transfected. The method of transfection is as follows:

55 μg each, of the DNA of the two plasmids (light chain and heavy chain carrying plasmids) was mixed and pre-incubated with Lipofectamine 2000 in DMEM Dulbecco's modified Eagle's medium) for 20 minutes. This mixture was then added to CHO cells growing in DMEM +10% serum and incubated at 37° C., 5% CO₂ for between 3 and 4 days.

Antibody was then purified from cell culture supernatant by Protein A chromatography using a loading buffer of 20 mM Phosphate pH 7. Purifications were performed using 1 ml HiTrap columns (Amersham, P# 17-5079-01) on an AKTA explorer HPLC. Proteins were eluted at low pH (0.1 M sodium citrate pH 3) and neutralised using 2 M Tris immediately after elution to around pH 7 to 7.2.

Material was then electrophoresed on SDS-PAGE. FIG. 4 confirms that the mutated heavy chain electrophoreses as a single species and hence the glycosylation site is mutated. This also confirms that Asn56 is at least partially glycosylated in the wild-type form.

Gene sequences containing the mutations N56S, N56Q, N56G and N56D as well as wild-type N56N were generated synthetically. In order to generate the remaining 15 amino acid mutations, the polymerase chain reaction (PCR) mutagenesis was employed.

Immediately following the codon coding for Asn56 in the codon optimised mutants is an XbaI restriction site. Oligonucleotides were synthesised which incorporate this XbaI site as well as codons coding for all remaining amino acids. These oligonucleotide sequences are shown in FIG. 14. These sequences are the complementary strand, and the correct codon sequence is also indicated in FIG. 14.

Oligonucleotides PP1 was synthesised (FIG. 14). This includes the HindIII restriction site at the start of the heavy chain DNA cassette, and also includes the Kozak sequence and the start of the coding region. PCR amplification was performed with PP1 and each of the mutant oligonucleotides, using the wild-type N56N expression vector as a template, to generate an approximately 230 base pair DNA fragment.

The fragments generated will incorporate the oligonucleotides used for the PCR priming, and hence include the requisite mutant codons. The fragments were then ligated back into the wild-type heavy chain vector from which the 230 bp HindIII-XbaI wild-type sequence fragment had been excised. In this way Asn56 was replaced by each of the remaining amino acids with no change to DNA or amino acid sequence other than at amino acid position 56. Sequence of the PCR amplified region was confirmed prior to any further analysis.

The molecular biology described above results in the light chain of HMFG1 cloned into pEE12.1 and the heavy chain of HMFG1 (and mutants) cloned into pEE6.4. It is also possible to ligate these into a single plasmid. This additional step was performed for some mutants (see below). Transfections can be performed equally well with light and heavy chains on either separate plasmids or on a single plasmid. In order to generate a single plasmid, both heavy and light chain expression plasmids were digested with NotI and BamHI restriction enzymes (the sites are shown in FIG. 12). This effectively linearises the light chain vector (with the loss of a very small DNA fragment). Digestion of the heavy chain plasmid results in the generation of a 3.7 kb fragment which contains the CMV promoter—heavy chain gene—polyA sequence. This 3.7 kb fragment was inserted into the linearised light chain plasmid to generate a single vector and this is shown in FIG. 13.

The following mutants were generated as a single plasmid (heavy and light chains on one vector): N56R, Q, E, G, H, I, L, K, M, F, P, S, Y, V

The following mutants were analysed with heavy and light chains still on separate plasmids: N56A, W, C, T, D

As stated previously, the expression of antibody chains from the same or separate plasmids will have no effect on antibody amino acid sequence, protein folding, antibody affinity or stability.

All mutant plasmids were transiently transfected into CHO cells as described previously and antibody was also purified as described previously. Multiple transient transfections and analyses were performed (up to 4 assays were performed on each mutant). This gave good data.

However stable cell lines were also generated with the mutants, and only data from these stable cell lines are presented here. However data from transient and stable transfections were essentially identical.

Mutant N56G was first transfected into NS0 cells and a stable cell line generated. Subsequently, all mutants (including the wildtype Asn and N56G) were transfected into the CHO K1SV cell line and stable cell lines were generated. The only exception was N56Y, which failed in the CHO K1SV eel line due to a technical error (and no opportunity to repeat this has occurred): data from transient transfection is however shown for this mutant.

Plasmids pEE12.1 and pEE6.4 and the glutamine synthetase selection method for generation of stable cell line are used under license from Lonza Biologics. Protocols for generation of stable cell lines were also supplied under license from Lonza Biologics, and these methods were followed for the current work.

In brief, NS0 cells were stably transfected with the N56G expression plasmid using Lipofectamine 2000. One day after transfection, cells were plated in wells of 96-well plates at either 50,000 cells/well, 10,000 cells/well or 2,000 cells/well. These were grown in glutamine-free DMEM +10% FCS until clones started to appear (approximately 3 weeks).

Mouse myeloma cell lines such as NS0 lack an endogenous glutamine synthetase gene, and are unable to grow in the absence of glutamine. The plasmid pEE12.1 contains a glutamine synthetase gene, and thus only cells which have this incorporated into the genome are able to grow. Outgrowing clones were expanded and antibody purified from the supernatant by Protein A affinity chromatography as described previously.

For transfection of the CHO K1SV cell line, plasmids were electroporated into the cells. Cells were then grown in CM2 medium, (obtained under license from Lonza Biologics) containing 10% FCS and 50 μM methionine sulphoximine (MSX). CHO cell lines contain a glutamine synthetase gene and hence are able to grow in the absence of glutamine. MSX is an inhibitor of glutamine synthetase, and only cells with additional GS activity from the transfected plasmids are able to grow. Following transfection, cells were inoculated into a tissue culture flask (and not plated in 96-well plates to generate clonal populations). In this way polyclonal stable cell lines were generated.

### Example 3

### Antigen Binding Activity

The protein concentration of samples was determined by an ELISA specific for human IgG (data not shown).

Material was then analysed for binding to antigen according to the following protocol:

96-well plates (Nunc Maxisorp) were coated with 100 μl of MUC1 at 1 μg/ml in carbonate-bicarbonate buffer (Sigma C3041) overnight at 4° C. MUC1 antigen used here was produced by the applicant and is a MUC1-GST fusion protein containing seven variable number tandem, repeat (VNTR) region; VNTR is the antigen recognised by MUC1.

Plates were then washed with a wash solution of PBS (phosphate buffered saline), +0.05% Tween 20, and serial dilutions of HMFG1 samples (wildtype protein or mutant) in incubation buffer (200 ml of PBS +0.2 g BSA (bovine serum albumin)) applied to the wells and incubated at 37° C. for 1 hour.

Plates were again washed, and treated with detection antibody (1.0 μl HRP (horse radish peroxidase) anti-human IgG (BD Pharmingen, P# 555788, or Jackson P#209-035-056) +1 ml incubation buffer) at 37° C. for 30 min, washed with wash buffer, and developed with TMB (tetramethylbenzidine) reagent (100 μl TMB (Sigma, T8665) warmed to room temperature and 100 μl sulphuric acid was used to stop the reaction after approximately 6 minutes). The optical density of the plates was read at 450 nm on a Spectromax plate reader immediately after development.

The optical density readings for N56G, N56D, N56S and N56Q are shown in FIG. 5. The N56G HMFG1 mutant exhibited an enhanced affinity for antigen in comparison to the wild type HMFG1. Initially, plasmids expressing mutants N56G, N56D, N56S and N56Q were generated. These were transiently transfected in CHO cells, and resulting antibody was analysed as described above.

Subsequently, polyclonal stable cell lines expressing all possible mutations were generated. FIGS. 15-18 show binding affinities of all mutant HMFG1 antibodies from these polyclonal cell lines (with, the exception of N56V; data for this is shown in FIG. 19 and is from transiently transfected cells). Included in these data are the wild-type N56N codon optimised plasmid, and the original non-codon-optimized HMFG1 (expressed from plasmid pAS6K). These two plasmids, which express wild-type Asn56 antibody, indicate the error intrinsic in such an analysis. In addition. FIG. 20 shows a comparison of N56G prepared, from the stable NS0 cell line compared to HMFG1 standard. This data for N56G using protein isolated from the NS0 cell line is very similar to that obtained, from the CHO cell line.

From these data an EC₅₀ value can be extracted which is a measure of affinity. These should always be compared to intra-assay controls due to inter-assay variation. FIG. 21 shows a table of the fold, difference in EC₅₀ of each mutant compared to HMFG1 standard. These data are extracted from FIGS. 15-20.

The three mutants with particularly good affinity to MUCI are in decreasing order: N56C>N56G>N56A. FIG. 21 shows that N56C has approximately 20-fold increased affinity, N56G approximately 10-fold increased affinity, and N56A approximately 5-fold increased affinity compared to HMFG1 standard.

It appears that no amino acid alteration at position 56 dramatically reduces affinity. Many mutants have approximately similar affinity compared with asparagine at this position. None of the mutated amino acids introduced are N-glycosylated, so all should reduce the glycan heterogeneity at this position. The marked increase in affinity with N56C is unexpected. The particularly good increase in affinity with N56G and N56A indicates that amino acids with small side chains are potentially preferred in this position.

### Example 4

### Antibody Dependent Cell Mediated Cytotoxicity

The N56G mutant prepared from the stable NS0 cell line in example 2 was compared to HMFG1 standard in an antibody dependent cell-mediated cytotoxicity (ADCC) assay. The HMFG1 standard was also produced from an NS0 cell line, and so glycans present in the antibody constant region are likely to be consistent between HMFG1 standard and N56G.

In brief, peripheral blood mononuclear cells (PBMCs) were isolated from whole blood from a healthy donor by centrifugation on a Ficoll-paque gradient. These are the effector cells. Target cells used were DU145 cells, which are known to express MUCI antigen on the cell, surface.

ADCC was performed using a DBLFIA cytotoxicity assay (Perkin-Elmer) in a 96-well plate. The method is based on loading target cells with an acetoxymethyl ester (BATDA) of a fluorescence enhancing ligand. When target cells are lysed, the released ligand is introduced to a europium solution to form a fluorescent chelate. The measured signal correlates directly with the amount of lysed cells.

The assay was performed essentially as described by the manufacturer. In brief, DU45 target cells were used at 5000 cells/well and PBMC effector cells at a 50:1 effector:target ratio. BATDA was pre-incubated with target cells for 15 minutes at 37° C., then washed. Effector cells, BATDA-loaded target cells and antibody (1 μg/ml) were mixed in wells of a 96-well plate and incubated for 2 hours at 37° C.

After this time, the plate was centrifuged and a sample of supernatant was removed from each well. This was incubated with europium for 20 minutes (according to Perkin-Elmer kit instructions), then fluorescence was measured on a Perkin Elmer Victor3 plate reader.

Background leakage of BATDA from target cells was measured, as was non-specific lysis of target cells by PBMCs in the absence of antibody. This background was subtracted from values obtained in test wells containing antibody. A measure of 100% lysis was determined by treating target cells with Triton-X 100, and lysis of test samples was calculated as % of 100% control.

This data shows that the N56G mutant was more potent at mediating HMFG1 dependent cytotoxicity than wildtype HMFG1.

### Example 5

### Association with Other Agents

The antibodies, antibody fragments and antibody derivatives of the invention may be conjugated to the other agents that they are associated with. Such immunoconjugates include an antibody conjugated to a cytotoxic agent (e.g. a chemotherapeutic agent) or a radioactive isotope.

Enzymatically active toxins and fragments thereof that can be used include diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta. et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

The antibody can be conjugated to a “receptor” (such streptavidin) for utilisation in tumour pre-targeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Cancer chemotherapeutic agents that the antibody/antibody fragments or antibody derivatives can be associated with (either by conjugation or otherwise) include: alkylating agents including nitrogen mustards such as mechlorethamine (HN₂), cyclophosphamide, ifosfamide, melphalan (L-sarcolysin) and chlorambucil; ethylenimines and methylmelamines such as hexarnethylmelamine, thiotepa; alkyl sulphonates such as busulfan; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); Anti-metabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin). Natural Products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; and biological response modifiers such, as interferon alphenomes. Miscellaneous agents including platinum co-ordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MIH); and adrenocortical suppressant such as mitotane (o,p′-DDD) aminoglutethimide; taxol and analogues/derivatives; and hormone agonists/antagonists such as flutamide and tamoxifen.

### Example 5

### Pharmaceutical Formulations and Administration

A further aspect of the invention provides a pharmaceutical formulation comprising a compound according to the first aspect of the invention in admixture with a pharmaceutically or veterinarily acceptable adjuvant, diluent or carrier.

Preferably, the formulation is a unit dosage containing a daily dose or unit, daily sub-dose or an appropriate fraction thereof, of the active ingredient.

The compounds of the invention will normally be administered orally or by any parenteral route, in the form of a pharmaceutical formulation comprising the active ingredient, optionally in the form of a non-toxic organic, or inorganic, acid, or base, addition salt, in a pharmaceutical acceptable dosage form. Depending upon the disorder and patient to be treated, as well as the route of administration, the compositions may be administered at varying doses.

In human therapy, the compounds of the invention can be administered alone but will generally be adminstered in admixture with a suitable pharmaceutical excipient diluent or carrier selected with regard to the intended route of administration and standard pharmaceutical practice.

For example, the compounds of the invention can be administered orally, buccally or sublingually in the form of tablets, capsules, ovules, elixirs, solutions or suspensions, which may contain flavouring or colouring agents, for immediate-, delayed- or controlled-release applications. The compounds of invention may also be administered via intracavernosal injection.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

The compounds of the invention can also be administered parenterally, for example, intravenously, intra-arterially, intraperitoneally, intrathecally, intraventricularly, intrasternally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient, and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For oral and parenteral administration to human patients, the daily dosage level of the compounds of the invention will usually be from 1 mg/kg to 30 mg/kg. Thus, for example, the tablets or capsules of the compound of the invention may contain a dose of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

The compounds of the invention can also be administered intranasally or by inhalation and are conveniently delivered in the form of a dry powder inhaler or an aerosol spray presentation from a pressurised container, pump, spray or nebuliser with the use of a suitable propellent, e.g. dichlorodifluoromethane, trichlorofluoromethane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator, may be formulated to contain a powder mix of a compound of the invention and a suitable powder base such as lactose or starch.

Aerosol or dry powder formulations are preferably arranged so that each metered dose or “puff” delivers an appropriate dose of a compound of the invention for delivery to the patient. It will be appreciated that the overall daily dose with an aerosol will vary from patient to patient, and may be administered in a single dose or, more usually, in divided doses though-out the day.

Alternatively, the compounds of the invention can be administered in the form of a suppository or pessary, or they may be applied topically in the form of a lotion, solution, cream, ointment or dusting powder. The compounds of the invention may also be transdermally administered, for example, by the use of a skin patch. They may also be administered by the ocular route, particularly for treating diseases of the eye.

For ophthalmic use, the compounds of the invention can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the compounds of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Formulations suitable for topical administration in the mouth include lozenges comprising the active ingredient in a flavoured basis, usually sucrose and acacia or tragacanth; pastilles-comprising the active ingredient in an inert basis such as gelatin and glycerin, or sucrose and acacia; and mouth-washes comprising the active, ingredient in a suitable liquid carrier.

Generally, in humans, oral or topical administration of the compounds of the invention is the preferred route, being the most convenient. In circumstances where the recipient suffers from a swallowing disorder or from impairment of drug absorption after oral administration, the drug may be administered parenterally, e.g. sublingually or buccally.

For veterinary use, a compound of the invention is administered as a suitably acceptable formulation in accordance with normal veterinary practice and the veterinary surgeon will determine the dosing regimen and route of administration which will be most appropriate for a particular animal.

1: A modified antibody molecule which selectively binds to a specific target,
wherein the modified antibody molecule is modified at, at least one amino acid residue that forms part of a glycosylation site in a variable region with respect to an unmodified parent antibody molecule,
wherein the modified antibody is not glycosylated at the modified glycosylation site, and
wherein the modified antibody exhibits a greater binding affinity for the specific target than the unmodified parent antibody molecule.

2: The modified antibody molecule as claimed in claim 1 wherein the at least one modified amino acid residue corresponds to an asparagine (N) in the unmodified parent antibody.

3: The modified antibody molecule as claimed in claim 1, wherein the site of the at least one modified amino acid residue corresponds to position 56 of SEQ ID NO: 6 or the corresponding residue in another antibody molecule.

4: The modified antibody molecule as claimed in claim 1, wherein the specific target is the MUC-I gene product.

5: The modified antibody molecule as claimed in claim 1, wherein the at least one modified amino acid residue is the amino acid, glycine.

6: The modified antibody molecule as claimed in claim 1, wherein the modified antibody molecule has an antigen binding selectivity equivalent to that of the anti-HMFG monoclonal antibody HMFG1.

7: The modified antibody molecule as claimed in claim 6 wherein the unmodified parent antibody is HMFG1.

8: The modified antibody molecule as claimed in claim 1, wherein the modified antibody molecule is a single chain antibody.

9: The modified antibody molecule as claimed in claim 8 wherein the single chain antibody is an ScFv.

10: The modified antibody molecule as claimed in claim 9 wherein the single chain antibody is a diabody.

11: The modified antibody molecule as claimed in claim 1, wherein the modified antibody molecule is humanised.

12: The modified antibody molecule as claimed in claim 1, wherein the modified antibody molecule is a chimeric antibody.

13: A nucleic acid having a nucleotide sequence encoding the modified antibody molecule of claim 1.

14: The nucleic acid of Claim 13 wherein the nucleotide sequence comprises a nucleotide sequence according to SEQ ID NO: 9, and wherein positions 232-234 of SEQ ID NO: 9 is a codon encoding any amino acid residue other than Asparagine.

15: An expression vector comprising a nucleotide sequence encoding the modified antibody molecule of claim 1.

16: The expression vector of Claim 15 wherein the nucleotide sequence comprises a nucleotide sequence according to SEQ ID NO: 9, and wherein positions 232-234 of SEQ ID NO: 9 is a codon encoding any amino acid residue other than Asparagine.

17: A host cell producing the modified antibody molecule claim 1, wherein the production of the modified antibody molecule results from the expression of a nucleotide sequence encoding the modified antibody molecule.

18: The host cell as claimed in claim 17 wherein the nucleotide sequence comprises a nucleotide sequence according to SEQ ID NO: 9, and wherein positions 232-234 of SEQ ID NO: 9 is a codon encoding any amino acid residue other than Asparagine.

19: The modified antibody molecule of claim 1, wherein the modified antibody molecule is associated with at least one other agent.

20: The modified antibody molecule as claimed in claim 19 wherein the at least one other agent is selected from the group consisting of: a drug, a toxin, a radionuclide, a nuclease, an enzyme, a cytokine, and a chemokine.

21: The modified antibody molecule as claimed in claim 20 wherein the at least one agent is conjugated to the modified antibody molecule.

22: The modified antibody as claimed in claim 20 wherein the at least one agent is a cytokine.

23: A composition comprising the modified antibody molecule of claim 1 and a pharmaceutically acceptable carrier, excipient and/or diluent.

24: A composition comprising the nucleic acid as claimed in claim 13 or 14.

25: The composition as claimed in claim 23 further comprising at least one other agent.

26: The composition as claimed in claim 25 wherein the at least one other agent is selected from the group consisting of: a drug, a toxin, a radionuclide, a nuclease, an enzyme, a cytokine, and a chemokine.

27: A method of treating, diagnosing and/or preventing a disease in a patient, the method comprising administering to the patient the modified antibody molecule of claim 1, wherein the disease is selected from the group consisting of: cancer, inflammatory disorders, cardiovascular diseases, infectious diseases, autoimmune disorders, central nervous system disorders, nephritis, sepsis, haemoglobinuria, chemotherapy induced thrombocytopenia, and addiction.

28: The method of claim 27 wherein the disease is Cancer.

29: The method of claim 28 wherein the cancer is breast cancer, ovarian cancer, uterine cancer, lung cancer, prostate cancer, colon cancer, B-NHL, multiple myeloma, AML, CLL or hairy cell leukaemia.

30: A phage containing the modified antibody nucleotide sequence of Claim 13 or 14.

31: The phage as claimed in claim 30 wherein the modified antibody nucleotide sequence is operably linked to a nucleotide sequence encoding a phage surface protein.

32: The phage as claimed in claim 30, wherein the phage is expressing and displaying the product of the modified antibody nucleotide sequence.

33. (canceled)

34: A method of performing a screening assay, the method comprising:
screening a library of phage in a phage display screening assay, and
identifying at least one phage in the library displaying antibodies, antibody fragments or antibody derivatives that are able to bind a target molecule.

35: The method of claim 34 wherein the target molecule is a MUC1 gene product.

36: A method of modifying an unmodified parent antibody molecule which binds to a specific target to produce a modified antibody, the method comprising:
substituting at least one amino acid residue that forms part of a glycosylation site in the variable region of the unmodified parent antibody molecule with a different amino acid residue, wherein the resulting modified antibody is not glycosylated at the modified glycosylation site, and wherein the resulting modified antibody exhibits a greater binding affinity for the specific target than the unmodified parent antibody molecule.

37-39. (canceled)


SEQUENCE LISTING (26 sequences)

SEQ ID NO: 1 - DNA, 732 bp, Homo sapiens
gccgccaccatgggctggagctgtatcatcctgttcctggtggccaccgccaccggcgtgcactccgacatccagatgacccagagcccaagcagcctgagcgccagcgtgggcgacagagtgaccatcacctgtaagtccagccagagcctgctgtactccagcaatcagaagatctacctggcctggtatcagcagaagccaggcaaggccccaaagctgctgatctactgggcctccaccagggagtctggcgtgccaagcagattcagcggctccggcagcggaaccgacttcaccttcaccatcagcagcctgcagccagaggacatcgccacctactactgccagcagtactacagataccctcggaccttcggccagggcaccaaggtggagatcaagagaaccgtggccgctccatctgtgttcatcttcccaccatctgatgagcagctgaagtctggcaccgctagcgtggtgtgcctgctgaataacttctaccccagagaggccaaggtgcagtggaaggtggacaacgccctgcagagcggcaactcccaggagagcgtgaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaacaggggcgagtgttgataa

SEQ ID NO: 2 - protein, 220 aa, Homo sapiens
DIQMTQSPSSLSASVGDRVTITCKSSQSLLYSSNQKIYLAWYQQKPGKAPKLLIYWASTRESGVPSRFSGSGSGTDFTFTISSLQPEDIATYYCQQYYRYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 1416 bp, Homo sapiens
gccgccaccatgggatggagctgtatcatcctgttcctggtggccacagctacaggcgtgcactcccaggtgcagctggtgcagtctggggccgaggtgaaaaagcctggggccagcgtgaaggtgtcctgcaaggcttctggctacaccttcagtgcctactggatcgagtgggtgagacaggctccaggaaagggcctggagtgggtgggagagattctgcctggaagtaataattctagatacaatgagaagttcaagggccgagtgacagtgactagagacacatccacaaacacagcctacatggagctgagcagcctgaggtctgaggacacagccgtgtattactgtgccagatcttacgactttgcctggtttgcttactggggccaggggactctggtgacagtgtccagcgcctccaccaagggcccatctgtgttccccctggctccatcctccaagagcacctctgggggcacagctgccctgggctgcctggtgaaggactacttccccgaacctgtgaccgtgagctggaactctggcgccctgaccagcggcgtgcacaccttccctgctgtgctgcagtccagcggactgtactccctgagcagcgtggtgaccgtgccctccagcagcctgggcacccagacctacatctgcaacgtgaatcacaagcccagcaacaccaaggtggacaagaaagtggagcccaaatcttgtgacaaaactcacacatgcccaccttgcccagctcctgaactgctggggggacctagcgtgttcctgttccccccaaaacccaaggacaccctgatgatctcccggacccctgaggtgacatgcgtggtggtggacgtgagccacgaagatcctgaggtgaagttcaactggtacgtggacggcgtggaggtgcacaatgccaagacaaagcctcgggaggagcagtacaacagcacctaccgggtggtgtctgtgctgaccgtgctgcaccaggactggctgaatggcaaggagtacaagtgcaaggtgtccaacaaagccctgccagcccccatcgagaaaaccatctccaaagccaaagggcagccccgggagccacaggtgtacaccctgcccccatccagagatgagctgaccaagaaccaggtgagcctgacctgtctggtgaaaggcttctatcccagcgacatcgccgtggagtgggagagcaatgggcagcccgagaacaactacaagaccacccctcccgtgctggactccgacggctccttcttcctgtactccaaactgaccgtggacaagagcaggtggcagcaggggaacgtgttcagctgctccgtgatgcatgaggctctgcacaaccactacacccagaagagcctgtccctgtctcctggaaaatgataa

SEQ ID NO: 4 - DNA, 1416 bp, Homo sapiens
  misc_feature (232)...(234): n= A, T, C or G
gccgccaccatgggatggagctgtatcatcctgttcctggtggccacagctacaggcgtgcactcccaggtgcagctggtgcagtctggggccgaggtgaaaaagcctggggccagcgtgaaggtgtcctgcaaggcttctggctacaccttcagtgcctactggatcgagtgggtgagacaggctccaggaaagggcctggagtgggtgggagagattctgcctggaagtnnnaattctagatacaatgagaagttcaagggccgagtgacagtgactagagacacatccacaaacacagcctacatggagctgagcagcctgaggtctgaggacacagccgtgtattactgtgccagatcttacgactttgcctggtttgcttactggggccaggggactctggtgacagtgtccagcgcctccaccaagggcccatctgtgttccccctggctccatcctccaagagcacctctgggggcacagctgccctgggctgcctggtgaaggactacttccccgaacctgtgaccgtgagctggaactctggcgccctgaccagcggcgtgcacaccttccctgctgtgctgcagtccagcggactgtactccctgagcagcgtggtgaccgtgccctccagcagcctgggcacccagacctacatctgcaacgtgaatcacaagcccagcaacaccaaggtggacaagaaagtggagcccaaatcttgtgacaaaactcacacatgcccaccttgcccagctcctgaactgctggggggacctagcgtgttcctgttccccccaaaacccaaggacaccctgatgatctcccggacccctgaggtgacatgcgtggtggtggacgtgagccacgaagatcctgaggtgaagttcaactggtacgtggacggcgtggaggtgcacaatgccaagacaaagcctcgggaggagcagtacaacagcacctaccgggtggtgtctgtgctgaccgtgctgcaccaggactggctgaatggcaaggagtacaagtgcaaggtgtccaacaaagccctgccagcccccatcgagaaaaccatctccaaagccaaagggcagccccgggagccacaggtgtacaccctgcccccatccagagatgagctgaccaagaaccaggtgagcctgacctgtctggtgaaaggcttctatcccagcgacatcgccgtggagtgggagagcaatgggcagcccgagaacaactacaagaccacccctcccgtgctggactccgacggctccttcttcctgtactccaaactgaccgtggacaagagcaggtggcagcaggggaacgtgttcagctgctccgtgatgcatgaggctctgcacaaccactacacccagaagagcctgtccctgtctcctggaaaatgataa

SEQ ID NO: 5 - DNA, 1416 bp, Homo sapiens
gccgccaccatgggatggagctgtatcatcctgttcctggtggccacagctacaggcgtgcactcccaggtgcagctggtgcagtctggggccgaggtgaaaaagcctggggccagcgtgaaggtgtcctgcaaggcttctggctacaccttcagtgcctactggatcgagtgggtgagacaggctccaggaaagggcctggagtgggtgggagagattctgcctggaagtggcaattctagatacaatgagaagttcaagggccgagtgacagtgactagagacacatccacaaacacagcctacatggagctgagcagcctgaggtctgaggacacagccgtgtattactgtgccagatcttacgactttgcctggtttgcttactggggccaggggactctggtgacagtgtccagcgcctccaccaagggcccatctgtgttccccctggctccatcctccaagagcacctctgggggcacagctgccctgggctgcctggtgaaggactacttccccgaacctgtgaccgtgagctggaactctggcgccctgaccagcggcgtgcacaccttccctgctgtgctgcagtccagcggactgtactccctgagcagcgtggtgaccgtgccctccagcagcctgggcacccagacctacatctgcaacgtgaatcacaagcccagcaacaccaaggtggacaagaaagtggagcccaaatcttgtgacaaaactcacacatgcccaccttgcccagctcctgaactgctggggggacctagcgtgttcctgttccccccaaaacccaaggacaccctgatgatctcccggacccctgaggtgacatgcgtggtggtggacgtgagccacgaagatcctgaggtgaagttcaactggtacgtggacggcgtggaggtgcacaatgccaagacaaagcctcgggaggagcagtacaacagcacctaccgggtggtgtctgtgctgaccgtgctgcaccaggactggctgaatggcaaggagtacaagtgcaaggtgtccaacaaagccctgccagcccccatcgagaaaaccatctccaaagccaaagggcagccccgggagccacaggtgtacaccctgcccccatccagagatgagctgaccaagaaccaggtgagcctgacctgtctggtgaaaggcttctatcccagcgacatcgccgtggagtgggagagcaatgggcagcccgagaacaactacaagaccacccctcccgtgctggactccgacggctccttcttcctgtactccaaactgaccgtggacaagagcaggtggcagcaggggaacgtgttcagctgctccgtgatgcatgaggctctgcacaaccactacacccagaagagcctgtccctgtctcctggaaaatgataa

SEQ ID NO: 6 - protein, 448 aa, Homo sapiens
QVQLVQSGAEVKKPGASVKVSCKASGYTFSAYWIEWVRQAPGKGLEWVGEILPGSNNSRYNEKFKGRVTVTRDTSTNTAYMELSSLRSEDTAVYYCARSYDFAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 448 aa, Homo sapiens
QVQLVQSGAEVKKPGASVKVSCKASGYTFSAYWIEWVRQAPGKGLEWVGEILPGSSNSRYNEKFKGRVTVTRDTSTNTAYMELSSLRSEDTAVYYCARSYDFAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 448 aa, Homo sapiens
QVQLVQSGAEVKKPGASVKVSCKASGYTFSAYWIEWVRQAPGKGLEWVGEILPGSGNSRYNEKFKGRVTVTRDTSTNTAYMELSSLRSEDTAVYYCARSYDFAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 448 aa, Homo sapiens
QVQLVQSGAEVKKPGASVKVSCKASGYTFSAYWIEWVRQAPGKGLEWVGEILPGSDNSRYNEKFKGRVTVTRDTSTNTAYMELSSLRSEDTAVYYCARSYDFAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 448 aa, Homo sapiens
QVQLVQSGAEVKKPGASVKVSCKASGYTFSAYWIEWVRQAPGKGLEWVGEILPGSQNSRYNEKFKGRVTVTRDTSTNTAYMELSSLRSEDTAVYYCARSYDFAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 27 bp, Artificial Sequence
  N56E
gtatctagaattctcacttccaggcag

SEQ ID NO: 12 - DNA, 27 bp, Artificial Sequence
  N56V
gtatctagaattaacacttccaggcag

SEQ ID NO: 13 - DNA, 27 bp, Artificial Sequence
  N56A
gtatctagaattagcacttccaggcag

SEQ ID NO: 14 - DNA, 27 bp, Artificial Sequence
  N56R
gtatctagaattcctacttccaggcag

SEQ ID NO: 15 - DNA, 27 bp, Artificial Sequence
  N56K
gtatctagaattcttacttccaggcag

SEQ ID NO: 16 - DNA, 27 bp, Artificial Sequence
  N56M
gtatctagaattcatacttccaggcag

SEQ ID NO: 17 - DNA, 27 bp, Artificial Sequence
  N56I
gtatctagaattaatacttccaggcag

SEQ ID NO: 18 - DNA, 27 bp, Artificial Sequence
  N56T
gtatctagaattagtacttccaggcag

SEQ ID NO: 19 - DNA, 27 bp, Artificial Sequence
  N56Y
gtatctagaattgtaacttccaggcag

SEQ ID NO: 20 - DNA, 27 bp, Artificial Sequence
  N56F
gtatctagaattaaaacttccaggcag

SEQ ID NO: 21 - DNA, 27 bp, Artificial Sequence
  N56H
gtatctagaattatgacttccaggcag

SEQ ID NO: 22 - DNA, 27 bp, Artificial Sequence
  N56L
gtatctagaattaagacttccaggcag

SEQ ID NO: 23 - DNA, 27 bp, Artificial Sequence
  N56P
gtatctagaattaggacttccaggcag

SEQ ID NO: 24 - DNA, 27 bp, Artificial Sequence
  N56W
gtatctagaattccaacttccaggcag

SEQ ID NO: 25 - DNA, 27 bp, Artificial Sequence
  N56C
gtatctagaattgcaacttccaggcag

SEQ ID NO: 26 - DNA, 31 bp, Artificial Sequence
  PP1
ggataagcttgccgccaccatgggatggagc